(19) United States
(12) Patent Application Publication
(10) Pub. No.: US 2023/0149696 A1
(43) Pub. Date: May 18, 2023

(54) Device Delivery Tool

(71) Applicant: Boston Scientific Scimed Inc., Maple Grove, MN (US)

(72) Inventors: Brady Scott Logan, Monticello, MN (US); Thomas P. Jancaric, Maple Grove, MN (US); Qian Liu, Plymouth, MN (US); Joel N. Groff, Delano, MN (US); Brian R. Reynolds, Ramsey, MN (US)

(21) Appl. No.: 17/989,033

(22) Filed: Nov. 17, 2022

Related U.S. Application Data
(60) Provisional application No. 63/280,357, filed on Nov. 17, 2021.

(51) Int. Cl. A61M 60/148; A61M 60/13

(52) U.S. Cl. CPC A61M 60/148; A61M 60/13

(57) ABSTRACT

A percutaneous circulatory support system includes a percutaneous circulatory support device including an impeller disposed within an impeller housing, the impeller being rotatable relative to the impeller housing to cause blood flow through the impeller housing. The system further includes a cannula coupled to the impeller housing, a cannula delivery tool configured for receiving and radially compressing the cannula, the cannula delivery tool having a proximal portion positioned adjacent a tapered portion, and a distal portion positioned adjacent the tapered portion.

### CROSS REFERENCE TO RELATED APPLICATION

This application claims priority to Provisional Application No. 63/280,357, filed Nov. 17, 2021, which is herein incorporated by reference in its entirety.

### TECHNICAL FIELD

The present disclosure relates to a device used to facilitate the intravascular delivery of a medical device. More specifically, the disclosure relates to percutaneous circulatory support systems having a cannula and a device to facilitate delivery of the cannula through delivery sheathing.

### BACKGROUND

Certain medical devices, such as circulatory support devices, are delivery intravascularly. The introduction of such devices to the vasculature often involves passing the devices through one or more delivery sheaths, and then guiding the device through the patient's vasculature to its final position. In the case of a circulatory support device to be placed in the left side of the heart, the device is commonly introduced into the femoral artery and passed through the vasculature until device enters the aorta. The cannula incorporated into the device is then passed through the aortic valve and into the left ventricle. Due to the size and construction of the such devices, and in particular the cannulas incorporated into circulatory support devices, passage of the devices through delivery sheaths may be difficult.

### SUMMARY

In Example 1, a percutaneous circulatory support system includes a device including a housing and a cannula coupled to the housing, a cannula delivery tool configured for receiving and radially compressing the cannula, the cannula delivery tool having a proximal portion positioned adjacent a tapered portion, and a distal portion positioned adjacent the tapered portion.

In Example 2, the system of Example 1 further includes wherein the cannula delivery tool is a laser cut tube and the cannula delivery tool comprises a plurality of closed cells.

In Example 3, the system of Example 1 further includes wherein the percutaneous circulatory support system includes a starter tube for receiving the cannula delivery tool and the cannula, wherein the cannula delivery tool is configured to compress when inserted into the starter tube.

In Example 4, the system of Example 3 further includes wherein the percutaneous circulatory support system further includes an introducer sheath, wherein the introducer sheath comprises an inner diameter, and when the cannula delivery tool is compressed, the cannula delivery tool is defined by an outer diameter that is less than the inner diameter of the introducer sheath.

In Example 5, the system of Example 1 further includes wherein the cannula delivery tool is composed of nitinol.

In Example 6, the system of Example 1 further includes wherein the proximal portion of the cannula delivery tool comprises a curved plate that extends on a first side of the cannula delivery tool, and wherein a tether extends from the curved plate and extends proximally to couple with the introducer sheath.

In Example 7, the system of Example 1 further includes wherein the cannula delivery tool includes a surface coating along the surface of the cannula delivery tool.

In Example 8, the system of Example 4 further includes wherein a coefficient of friction between the cannula and the introducer sheath is greater than a coefficient of friction between the cannula delivery tool and the introducer sheath.

In Example 9, the system of Example 6 further includes wherein the system further comprises a guidewire that extends within the cannula delivery tool and the cannula, and wherein the guidewire extends through a second side of the cannula delivery tool, the second side being opposite the first side relative to a longitudinal axis of the cannula delivery tool.

In Example 10, a method of deploying a percutaneous device includes providing a percutaneous support system including the percutaneous device having at least a housing coupled to a cannula, a handle for actuation of the percutaneous support system, a starter tube loaded on a catheter of the percutaneous support system, a cannula delivery tool loaded onto the catheter, and an introducer sheath. The method further includes extending the cannula delivery tool over the cannula, retracting the cannula delivery tool and the percutaneous support device into the starter tube and inserting the starter tube at least partially into the introducer sheath. The method further includes extending the cannula delivery tool out of a distal portion of the starter tube and at least partially out of a distal portion of the introducer sheath and extending the cannula out of the cannula delivery tool.

In Example 11, the method of Example 10 further includes wherein retracting the delivery tool into the starter tube compresses cannula delivery tool such that the cannula delivery tool comprises an outer diameter that is less than an inner diameter of the introducer sheath.

In Example 12, the method of Example 10 further includes wherein the percutaneous support system further includes a guidewire, and wherein the method further includes extending the guidewire through the cannula delivery tube and the percutaneous support device prior to extending the cannula delivery tool over the cannula.

In Example 13, the method of Example 12 further includes wherein the percutaneous support system further comprises a tether coupled to the introducer sheath and the cannula delivery tool, such that a length of the tether dictates how far the cannula delivery tool extends out of the introducer sheath.

In Example 14, the method of Example 10 further includes wherein the method further comprises retracting the tether in order to retract the cannula delivery tool through the introducer sheath and into the starter tube.

In Example 15, the method of Example 11 further includes wherein the cannula delivery tool comprises a tapered portion and a surface treatment, both configured to allow the cannula delivery tool to compress when retracted into the start tube.

In Example 16, a percutaneous circulatory support system includes a percutaneous circulatory support device including an impeller disposed within an impeller housing, the impeller being rotatable relative to the impeller housing to cause blood flow through the impeller housing. The system further includes a cannula coupled to the impeller housing, a cannula delivery tool configured for receiving and radially compressing the cannula, the cannula delivery tool having a proximal portion positioned adjacent a tapered portion, and a distal portion positioned adjacent the tapered portion.

In Example 17, the system of Example 16 further includes wherein the cannula delivery tool is a laser cut tube and the cannula delivery tool comprises a plurality of closed cells.

In Example 18, the system of Example 16 further includes wherein the percutaneous circulatory support system includes a starter tube for receiving the cannula delivery tool and the cannula, wherein the cannula delivery tool is configured to compress when inserted into the starter tube.

In Example 19, the system of Example 18 further includes wherein the percutaneous circulatory support system further includes an introducer sheath, wherein the introducer sheath comprises an inner diameter, and when the cannula delivery tool is compressed, the cannula delivery tool is defined by an outer diameter that is less than the inner diameter of the introducer sheath.

In Example 20, the system of Example 16 further includes wherein the cannula delivery tool is composed of nitinol.

In Example 21, the system of Example 16 further includes wherein the proximal portion of the cannula delivery tool comprises a curved plate that extends on a first side of the cannula delivery tool, and wherein a tether extends from the curved plate and extends proximally to couple with the introducer sheath.

In Example 22, the system of Example 21 further includes wherein the system further comprises a guidewire that extends within the cannula delivery tool and the cannula, and wherein the guidewire extends through a second side of the cannula delivery tool, the second side being opposite the first side relative to a longitudinal axis of the cannula delivery tool.

In Example 23, the system of Example 16 further includes wherein the cannula delivery tool comprises a surface coating along the surface of the cannula delivery tool.

In Example 24, the system of Example 23 further includes wherein the surface coating is silicone.

In Example 25, the system of Example 19 further includes wherein a coefficient of friction between the cannula and the introducer sheath is greater than a coefficient of friction between the cannula delivery tool and the introducer sheath.

In Example 26, a cannula delivery tool configured for delivering a cannula includes a proximal portion opposite a distal portion and a body portion extending therebetween, wherein the body portion comprises a tapered portion, a curved plate extending from the proximal portion configured for easing the introduction of the cannula delivery tool into a sheath, and a plurality of closed cells along the body portion formed by laser cutting a tube that forms the cannula delivery tool.

In Example 27, the cannula delivery tool of Example 26 further includes herein a tether is coupled to the curved plate and extends proximally from the curved plate of the cannula delivery tool, and wherein the tether is welded to the curved plate.

In Example 28, the cannula delivery tool of Example 26 further includes wherein the cannula delivery tool is composed of one of nitinol and stainless steel, and wherein the cannula delivery tool comprises a surface treatment.

In Example 29, the cannula delivery tool of Example 26 further includes wherein the cannula delivery tool is configured to compress from an expanded configuration having an expanded outer diameter to a compressed configuration have a compressed outer diameter.

In Example 30, a method of deploying a percutaneous support device includes providing a percutaneous support system including the percutaneous support device having at least an impeller housing coupled to a cannula, a handle for actuation of the percutaneous support system, a starter tube loaded on a catheter of the percutaneous support system, a cannula delivery tool loaded onto the catheter, and an introducer sheath. The method further includes extending the cannula delivery tool over the cannula, retracting the cannula delivery tool and the percutaneous support device into the starter tube and inserting the starter tube at least partially into the introducer sheath. The method further includes extending the cannula delivery tool out of a distal portion of the starter tube and at least partially out of a distal portion of the introducer sheath and extending the cannula out of the cannula delivery tool.

In Example 31, the method of Example 30 further includes wherein retracting the delivery tool into the starter tube compresses cannula delivery tool such that the cannula delivery tool comprises an outer diameter that is less than an inner diameter of the introducer sheath.

In Example 32, the method of Example 30 further includes wherein the percutaneous support system further includes a guidewire, and wherein the method further includes extending the guidewire through the cannula delivery tube and the percutaneous support device prior to extending the cannula delivery tool over the cannula.

In Example 33, the method of Example 32 further includes wherein the percutaneous support system further comprises a tether coupled to the introducer sheath and the cannula delivery tool, such that a length of the tether dictates how far the cannula delivery tool extends out of the introducer sheath.

In Example 34, the method of Example 30 further includes wherein the method further comprises retracting the tether in order to retract the cannula delivery tool through the introducer sheath and into the starter tube.

In Example 35, the method of Example 31 further includes wherein the cannula delivery tool comprises a tapered portion and a surface treatment, both configured to allow the cannula delivery tool to compress when retracted into the start tube.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a diagram of a circulatory support system, in accordance with embodiments of the subject matter disclosed herein.

FIG. 2A illustrates a sectional view of a portion of the circulatory support system, in accordance with embodiments of the subject matter disclosed herein.

FIG. 2B illustrates a sectional view of a portion of the circulatory support system, in accordance with embodiments of the subject matter disclosed herein.

FIG. 3 illustrates a top view of a cannula delivery tool, in accordance with embodiments of the subject matter disclosed herein.

FIG. 4 illustrates a side view of the cannula delivery tool of FIG. 3, in accordance with embodiments of the subject matter disclosed herein.

FIG. 5 illustrates an enlarged top view of a portion of the cannula delivery tool of FIG. 3, in accordance with embodiments of the subject matter disclosed herein.

FIG. 6 illustrates an enlarged view of a portion of the cannula delivery tool of FIG. 3, in accordance with embodiments of the subject matter disclosed herein.

FIG. 7 is a flow chart of a method of delivering a circulatory support device, in accordance with embodiments of the subject matter disclosed herein.

### DETAILED DESCRIPTION

Various modifications and additions can be made to the exemplary embodiments discussed without departing from the scope of the present invention.

For example, while the embodiments described above refer to particular features, the scope of this invention also includes embodiments having different combinations of features and embodiments that do not include all of the above described features.

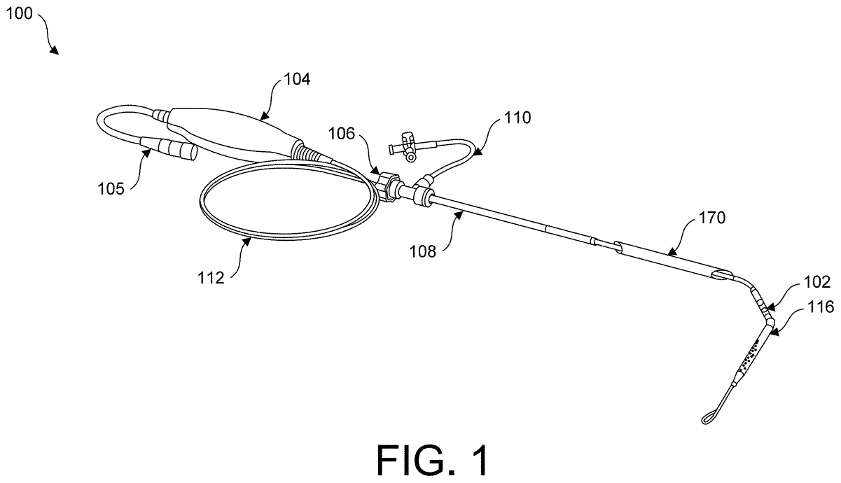

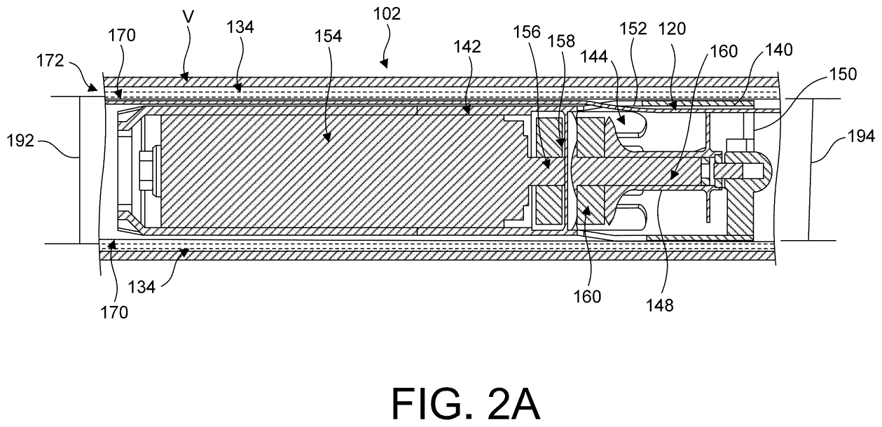

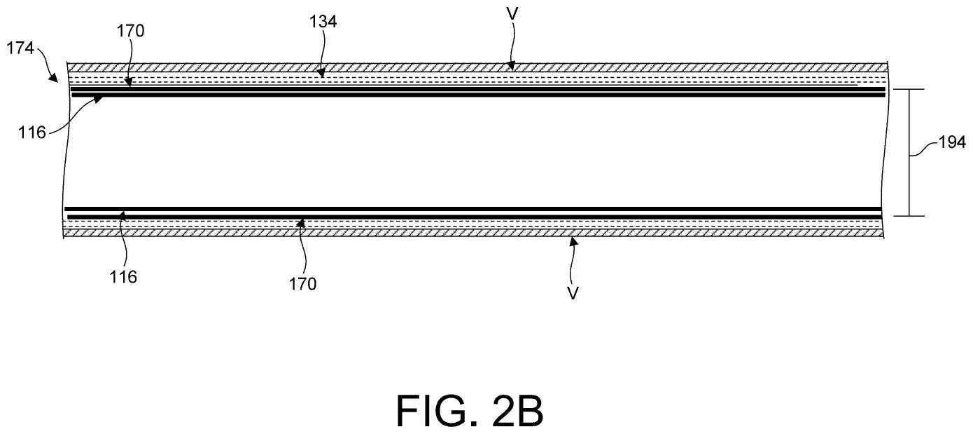

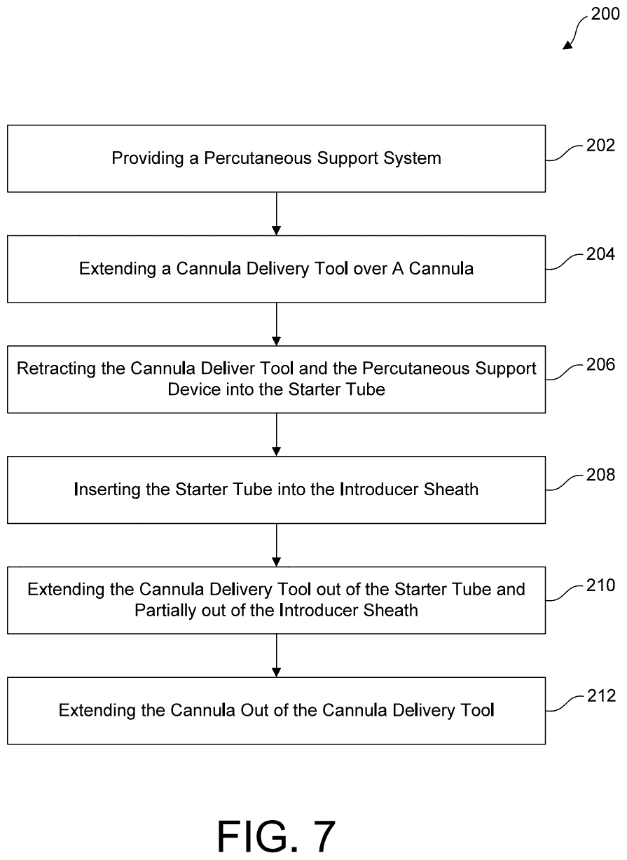

FIG. 1 depicts a side sectional view of several components of an illustrative percutaneous circulatory support system 100 in accordance with embodiments of the subject matter disclosed herein. Generally, the system 100 includes a percutaneous circulatory support device 102 (also referred to herein, interchangeably, as a “blood pump”), a cannula 116, and an introducer sheath 134 (shown in FIG. 2A). In some embodiments, the system 100 may also include a guidewire 120 (shown in FIG. 2A). As described in further detail below with reference to FIG. 7, the introducer sheath 134 facilitates percutaneous delivery of the blood pump 102 and the cannula 116, to a target location within a patient, such as within the patient's heart.

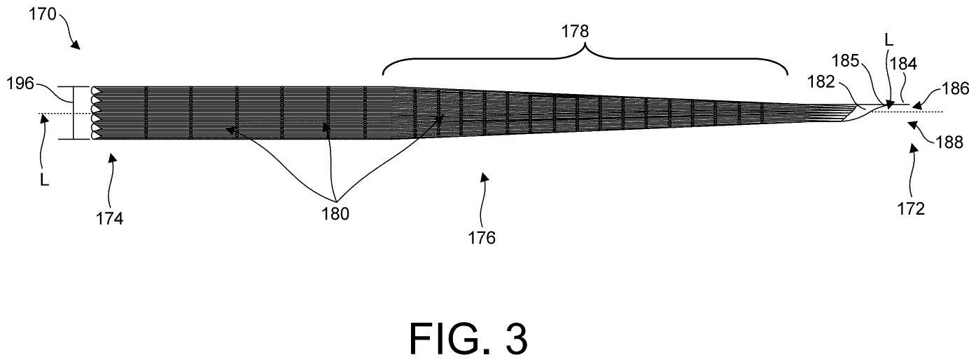

With continued reference to FIG. 1, the system 100 also includes a handle 104 connected to a starter tube hemostatic valve 106. The starter tube hemostatic valve 106 is coupled to both a starter tube 108 and a starter tube flushing line 110. The handle 104 may also couple with a proximal catheter 112. The starter tube 108 is illustrated as coupled to a cannula delivery tool 170 which is configured to receive the cannula 116 prior to and during delivery of the blood pump 102 and the cannula 116, as will be described further herein. In these embodiments, at least handle 104, starter tube 108, starter tube hemostatic valve 106, introducer sheath 134 (FIG. 3) and the cannula delivery tool 170 are configured for delivering the blood pump 102 and the cannula 116, to the target location with the patient, as will be described further herein. In some embodiments, the guidewire 120 (FIG. 2) may also be used to facilitate the delivery of the percutaneous circulatory support device 102. While described herein with reference to the percutaneous circulatory support system 100, cannula delivery tool 170 may be used for delivery of various other types of medical devices and is not limited to the exampled described herein.

As illustrated in FIG. 2A, the blood pump 102 generally includes an impeller assembly housing 140 and a motor housing 142. In some embodiments, the impeller assembly housing 140 and the motor housing 142 may be integrally or monolithically constructed. In other embodiments, the impeller assembly housing 140 and the motor housing 142 may be separate components configured to be removably or permanently coupled.

The impeller assembly housing 140 carries an impeller assembly 144 therein. The impeller assembly 144 includes an impeller shaft 146 and an impeller 148 that rotates relative to the impeller assembly housing 140 to drive blood through the blood pump 102. More specifically, the impeller 148 causes blood to flow from a blood inlet 150 formed on the impeller assembly housing 140, through the impeller assembly housing 140, and out of a blood outlet 152 formed on the impeller assembly housing 140. In some embodiments the impeller shaft 146 and the impeller 148 may be integrated, and in other embodiments the impeller shaft 146 and the impeller 148 may be separate components. As shown in FIGS. 1 and 2, the inlet 150 and/or the outlet 152 may each include multiple apertures. In other embodiments, the inlet 150 and/or the outlet 152 may each include a single aperture. As shown in FIG. 2A, the inlet 150 may be formed on an end portion of the impeller assembly housing 140 and the outlet 152 may be formed on a side portion of the impeller assembly housing 140. In other embodiments, the inlet 150 and/or the outlet 152 may be formed on other portions of the impeller assembly housing 140. In some embodiments, the impeller assembly housing 140 may couple to a distally extending cannula (not shown), and the cannula may receive and deliver blood to the inlet 150.

With continued reference to FIG. 2A, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components.

In some embodiments, a controller (not shown) may be operably coupled to the motor 154 and configured to control the motor 154. In some embodiments, the controller may be disposed within the motor housing 142. In other embodiments, the controller may be disposed outside of the motor housing 142 (for example, in a catheter handle, an independent housing, etc.). In some embodiments, the controller may include multiple components, one or more of which may be disposed within the motor housing 142. According to embodiments, the controller may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more central processing units (CPUs), software, hardware, firmware, or any combination of these and/or other components. Although the controller is referred to herein in the singular, the controller may be implemented in multiple instances, distributed across multiple computing devices, instantiated within multiple virtual machines, and/or the like. In other embodiments, the motor 154 may be controlled in other manners.

FIG. 2A illustrates a partial sectional view of various components of the system 100 after insertion into a blood vessel V, the process of which will be described further with reference to FIG. 7. Specifically, FIG. 2A illustrates the proximal portion 172 of the cannula delivery tool 170 and the percutaneous circulatory support device 102 positioned within the introducer sheath 134, such that the introducer sheath 134 surrounds the cannula delivery tool 170 during at least a portion of the delivery process of blood pump 102. For example, the introducer sheath 134 has an inner diameter 192 that is larger than a compressed outer diameter 194 of the cannula delivery tool 170 such that cannula delivery tool 170 fits entirely circumferentially within the introducer sheath 134. In this way, the cannula delivery tool 170 is positioned between the introducer sheath 134 and at least a portion of the percutaneous circulatory device 102.

FIG. 2B illustrates a partial sectional view of various components of the system 100 after insertion into the blood vessel V. Specifically, FIG. 2B illustrates the distal portion 174 of the cannula delivery tool 170 compressed onto the cannula 116 and the cannula delivery tool 170 positioned within the introducer sheath 134. Similar to the illustrative embodiment of FIG. 2A, the cannula deliver tool 170 is configured to have the compressed outer diameter 194. In this way, the cannula delivery tool 170 is positioned between the introducer sheath 134 and the cannula 116, and therefore, the cannula 116 does not come into direct contact with the introducer sheath 134. This may provide the benefit of reducing the frictional force between the introducer sheath 134 and the cannula 116 that would otherwise occur, as will be described further herein. While the cannula 116 and the cannula delivery tool 170 are described with reference to a use with the percutaneous circulatory support device 102, various other percutaneous circulatory support devices that may be different than device 102 may be used. Further, the cannula 116 and the cannula delivery tool 170 may be used with any variety of percutaneous devices and delivery systems. The cannula delivery tool 170 will be described further in detail herein.

FIG. 3 illustrates a top view of the cannula delivery tool 170 in an expanded configuration having an expanded outer diameter 196 that is larger than the compressed outer diameter 194 as illustrated in the configuration of FIG. 2. Additionally, the cannula delivery tool 170 comprises a proximal portion 172, a distal portion 174, and a body portion 176 extending between the proximal portion 172 and the distal portion 174 along a longitudinal axis L. At least a section of the body portion 176 comprises a tapered portion 178 which may be configured to aid in the retraction of the cannula delivery tool 170 into the starter tube 108 (FIG. 1), as will be described further with reference to FIG. 7. Additionally, the body portion 176 of the cannula delivery tool 170 is defined by a plurality of closed cells 180 that have been machined into the cannula delivery tool 170. In some embodiments, the cannula delivery tool 170 is composed of a metal material, such as nitinol or stainless steel, that has been laser cut to form the plurality of closed cells 180. The use of laser cutting or otherwise machining the plurality of closed cells 180 allows for openings to be formed into the cannula delivery tool 170 that may contribute to the ability of the cannula delivery tool 170 to be compressible and expandable, while still minimizing a thickness of the cannula delivery tool 170. For example, in conventional methods a braided design may be used for creating a plurality of closed cells in a delivery tool. However, the braided design increases the thickness of the delivery tool as the material has to weave and form intersections wherein some of the material is stacked on top of one another. This may be a disadvantage if the delivery tool needs to be positioned within an additional structure, for example, as is the case with the cannula delivery tool 170 described herein. Additionally, in some embodiments, the cannula delivery tool 170 has a surface coating such as silicone or PET to optimize the surface properties during delivery, as will be described further with reference to FIG. 7. In further embodiments, the surface coating may be any other lubricious coating or surface treatment which may reduce a coefficient of the cannula delivery tool 170.

With continued reference to FIG. 3, the proximal portion 172 comprises a plate 182. As illustrated, the plate 182 is curved and extends proximally of the cannula delivery tool 170 and couples to a tether 184. The plate 182 may be laser cut from the cannula delivery tool 170 or otherwise machined. The plate 182 is shown having an apex 185 that extends out onto a first side 186 of the device, the first side 186 defined as a first side 186 of the device relative to the longitudinal axis L of the cannula delivery tool 170. The first side 186 may be referred to as an upper side of the cannula delivery tool 170. While the embodiments herein are illustrated comprising the plate 182 at the proximal portion 172, in other embodiments, the cannula delivery tool 170 does not comprise the plate 182. In these embodiments, the delivery tool 170 may have an end formed similar to the configuration of the distal portion 174 and/or the configuration of the body portion 176.

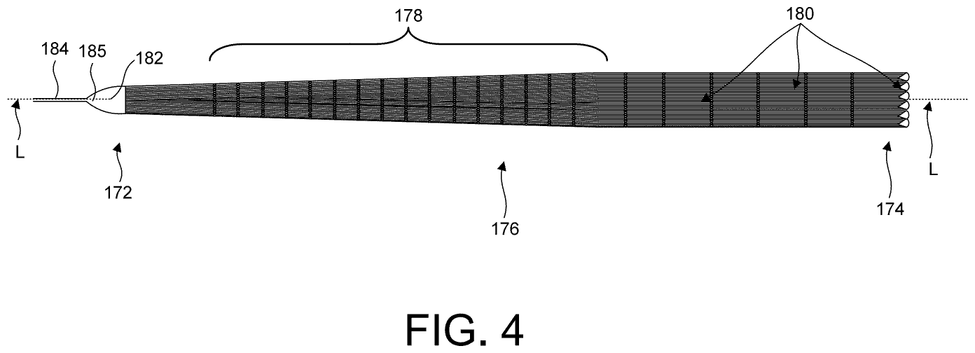

FIG. 4 illustrates a side view of the cannula delivery tool 170 of FIG. 3, showing the tether 184 extending proximally from the plate 182. In various embodiments, the tether 184 may be a separate wire that is welded to the plate 182. When welded, the tether 184 may comprise a weld protecting surface coating to increase the stability of the tether 184. For example, a heat shrink may be incorporated about the tether 184. In further embodiments, the tether 184 may be formed through laser cutting an original tube that forms the cannula delivery tool 170. In these embodiments, there may be an advantage provided in that the tether 184 is formed in one piece with the cannula delivery tool 170 and likelihood of breakage at the coupling point is reduced. Various other embodiments of the tether may be incorporated, and further methods of manufacture may be imagined.

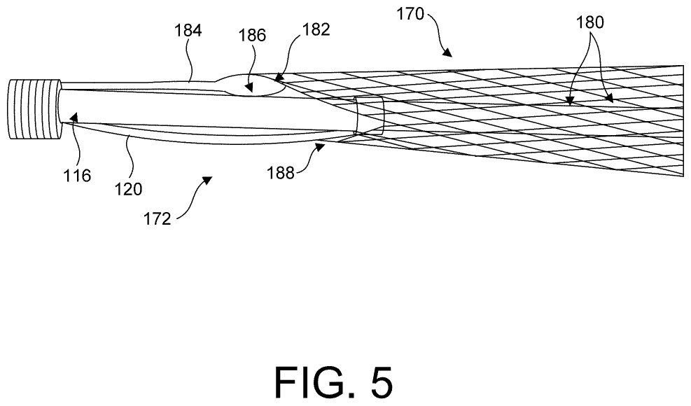

FIG. 5 illustrates an enlarged and sectional view of the proximal portion 172 of the cannula delivery tool 170 having the plate 182 coupled to the tether 184. The tether 184 extends along the same side as the plate 182, illustratively the first side 186. The tether 184 may additionally comprise radiopaque markers to aid the physician in identifying the positioning of the tether 184, and thus the cannula delivery tool 170, during delivery. As previously disclosed, the guidewire 120 may be used in combination with the illustrative percutaneous circulatory support system 100 for delivering the percutaneous circulatory support device 102 and the cannula 116. For example, FIG. 5 illustrates the cannula delivery tool 170 in use with the guidewire 120. Due to the configuration of plate 182 extending only on the first side 186 of the cannula delivery tool 170, the guidewire 120 has an open and relatively non-impeded passageway into the cannula delivery tool 170 on a second side 188 of the cannula delivery tool 170. The second side 188 is positioned opposite to the first side 186 relative to the longitudinal axis L. This may increase the ease with which the guidewire 120 may pass through the cannula delivery tool 170 as there is an integrated opening in the cannula delivery tool 170 formed for the guidewire 120 and the interaction between the guidewire 120, the cannula 116 and the cannula delivery tool 170 during insertion of the guidewire 120 can be reduced. However, as previously described, the illustrative percutaneous circulatory support system 100 can be used without the guidewire 120 as well.

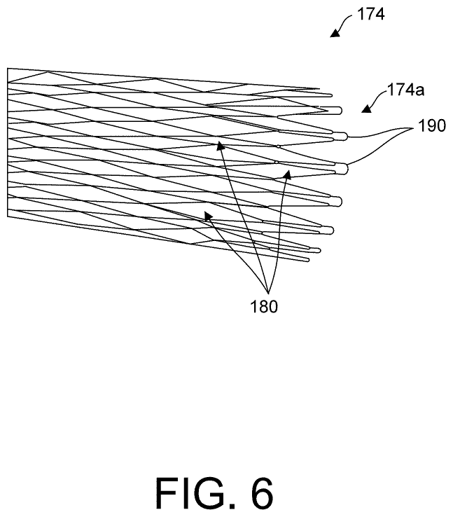

Further, FIG. 6 illustrates the distal portion 174 of the cannula delivery tool 170 in more detail. Specifically, the distal portion 174 of the cannula delivery tool 170 may be designed such that it provides an atraumatic end 174a, thus functioning to reduce harsh and/or sharp impact, or any damage to the vasculature by the cannula delivery tool 170 during delivery. Additionally, the cannula delivery tool 170 may not dislodge calcification that may be present on a vessel wall if the distal portion 174 includes an atraumatic end 174a. For example, the illustrative embodiment of FIG. 6 shows various configurations of the plurality of closed cells 180 having rounded apices 190 that extend distally from the distal portion 174. The rounded apices 190 provide the benefit of a less harsh or sharp interface than would otherwise be present if a jutted or pointed end were incorporated. While illustrated as having rounded apices 190, the distal portion 174 may be varied in configuration to provide this atraumatic end 174a.

FIG. 7 illustrates a method 200 of delivering a percutaneous circulatory support device, for example the percutaneous circulatory support device 102 described with reference to FIGS. 1 and 2, using cannula delivery tool 170. The following method 200 will be described with reference to FIG. 7 and the components as illustrated in at least FIGS. 1 and 2.

At block 202, the method first comprises providing a percutaneous support system, for example, the percutaneous circulatory support system 100. Further, at block 204, the method 200 includes extending the cannula delivery tool 170 over the cannula 116 of the illustrative percutaneous circulatory support system 100. Extending the cannula delivery tool 170 over the cannula 116 may include positioning the cannula delivery tool 170 such that the entire cannula 116 is enclosed by the cannula delivery tool 170. During this step, the cannula delivery tool 170 is in an expanded configuration such that the cannula delivery tool 170 has the expanded outer diameter 196 (FIG. 3) to facilitate positioning over the cannula 116.

Additionally, the method 200 comprises the step illustrated at block 206 of retracting the cannula delivery tool 170 and the percutaneous circulatory support device 102 into the starter tube 108. As such, the cannula delivery tool 170, the cannula 116 and the percutaneous circulatory support device 102 are retracted into the starter tube 108 to prepare for insertion into the body of a patient. During this step, the cannula delivery tool 170 is configured to compress from the expanded outer diameter 196 (FIG. 3) to the compressed outer diameter 194 (FIG. 2A and FIG. 2B) such that the cannula delivery tool 170 fits within the starter tube 108 and eventually the introducer sheath 134. The ability of the cannula delivery tool 170 to compress to have the compressed outer diameter 194 is at least in part due to the curved shape of the plate 182, the tapered portion 178 of the cannula delivery tool 170, and the material forming the cannula delivery tool 170. During this step, the cannula delivery tool 170 may be compressed down to the compressed outer diameter 194 such that cannula delivery tool 170 is in direct contact with the cannula 116.

At block 208, the method 200 includes inserting the starter tube 108, which includes the cannula delivery tool 170 and circulatory support device 102, into the introducer sheath 134. In various embodiments, the introducer sheath 134 has already been positioned at least partially into an artery of the patient, for example the femoral artery, of the patient.

At block 210, the method 200 further includes extending the cannula delivery tool 170 out of the starter tube 108 and partially out of the introducer sheath 134. This step further includes wherein the starter tube 108 is not extended entirely out of a distal portion of the introducer sheath 134, such that when the cannula delivery tool 170 is pushed out of the starter tube 108, the cannula delivery tool 170 is in direct contact with the interior of the introducer sheath 134, for example as shown in FIG. 2. As a result of the cannula delivery tool 170 being positioned at the compressed outer diameter 194, the cannula delivery tool 170 is already configured to fit within the introducer sheath 134 when deployed from the starter tube 108. As previously described, cannula delivery tool 170 includes a surface coating that may increase the ease with which the cannula delivery tool 170 slides within the introducer sheath 134. Additionally, the laser cut plurality of closed cells 180 formed into the cannula delivery tool 170, as opposed to the use of a braided structure, may also increase the ease with which that the cannula delivery tool 170 slides within the cannula delivery tool 170, as the cannula delivery tool 170 comprises one main layer of material, rather than stacked intersections of material. More specifically, the cannula delivery tool 170 may have a coefficient of friction when in contact with the introducer sheath 134 that is less than the coefficient of friction that would result between contact of the introducer sheath 134 and the cannula 116. In this way, the use of the cannula delivery tool 170 provides an advantage to the illustrative percutaneous circulatory support system 100 in that there is less friction during the deployment of the cannula 116 through the introducer sheath 134, which may otherwise cause deficiencies in the delivery process, including malfunctions and damage to the components.

With reference again to FIG. 7, during the step at block 210, the cannula delivery tool 170, which is still surrounds the cannula 116, is extended out of the distal portion of the introducer sheath 134, however, the extent that which the cannula delivery tool 170 is extended is limited by the tether 184, as the tether 184 couples the cannula delivery tool 170 and the introducer sheath 134. The tether 184 also allows for the ability to retract the cannula delivery tool 170 back into the introducer sheath 134 when desired. As the cannula delivery tool 170 extends out of the distal portion of the introducer sheath 134, the cannula delivery tool 170 expands radially. In various embodiments, the cannula delivery tool 170 expands radially such that the cannula delivery tool 170 is defined by the expanded outer diameter 196 again.

The method 200 then comprises the step at block 212, including extending the cannula 116 out of the cannula delivery tool 170. During this step, the cannula 116 and thus the percutaneous circulatory support device 102 that is coupled with the cannula 116, are released from the cannula delivery tool 170. The circulatory support device 102 can then be deployed through the patient's vasculature and into the heart of the patient. Once the circulatory support device 102 passes out of the cannula delivery tool 170, the cannula delivery tool 170 may be retracted into the introducer sheath 134 and further into the starter tube 108. When retracted into the introducer sheath 134, the diameter of the cannula delivery tool 170 is compressed down, as described further herein.

Further, in various embodiments, the illustrative percutaneous circulatory support system 100 may include the guidewire 120 for use in deploying the cannula 116 and the 102. For example, in these embodiments, prior to extending the cannula delivery tool over the cannula, the method 200 includes extending the guidewire 120 through the cannula delivery tool 170, specifically through the first side 186 as discussed with reference to FIG. 5, and through the percutaneous circulatory support device 102. The guidewire 120 may be used to aid in the delivery and the positioning of the cannula 116 and percutaneous circulatory support device 102.

Additionally, in embodiments, the method 200 may include retracting the tether 184 to retract the cannula delivery tool 170 through the introducer sheath 134 and into the starter tube 108 for removal. This step is optimized by the curved shape of the plate 182 of the cannula delivery tool 170, specifically in that the curved shape of the plate 182 allows for the cannula delivery tool 170 to be more easily captured back into the introducer sheath 134 and compressed back down to the compressed outer diameter 196 (FIG. 2A and FIG. 2B).

While the cannula delivery tool 170 and the cannula 116 are described throughout as begin used with a percutaneous circulatory support system 100 for delivering the percutaneous circulatory support device 102, the cannula delivery tool 170 and the cannula 116 may be used with a variety of different systems. Specifically, the cannula delivery tool 170 may also be used with a variety of the different medical devices, such as devices including balloon, stents, or other radially compressible and expandable devices that are introduced intravascularly. The embodiments described herein are not meant to be limiting and are provided as an example thereof.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A percutaneous circulatory support system, comprising:
a percutaneous circulatory support device including an impeller disposed within an impeller housing, the impeller being rotatable relative to the impeller housing to cause blood flow through the impeller housing;
a cannula coupled to the impeller housing,
a cannula delivery tool configured for receiving and radially compressing the cannula, the cannula delivery tool having a proximal portion positioned adjacent a tapered portion, and a distal portion positioned adjacent the tapered portion.

2. The percutaneous circulatory support system of claim 1, wherein the cannula delivery tool is a laser cut tube and the cannula delivery tool comprises a plurality of closed cells.

3. The percutaneous circulatory support system of claim 1, wherein the percutaneous circulatory support system includes a starter tube for receiving the cannula delivery tool and the cannula, wherein the cannula delivery tool is configured to compress when inserted into the starter tube.

4. The percutaneous circulator support system of claim 3, wherein the percutaneous circulatory support system further includes an introducer sheath, wherein the introducer sheath comprises an inner diameter, and when the cannula delivery tool is compressed, the cannula delivery tool is defined by an outer diameter that is less than the inner diameter of the introducer sheath.

5. The percutaneous circulatory support system of claim 1, wherein the cannula delivery tool is composed of nitinol.

6. The percutaneous circulatory support system of claim 1, wherein the proximal portion of the cannula delivery tool comprises a curved plate that extends on a first side of the cannula delivery tool, and wherein a tether extends from the curved plate and extends proximally to couple with the introducer sheath.

7. The percutaneous circulatory support system of claim 6, wherein the system further comprises a guidewire that extends within the cannula delivery tool and the cannula, and wherein the guidewire extends through a second side of the cannula delivery tool, the second side being opposite the first side relative to the longitudinal axis.

8. The percutaneous circulatory support system of claim 1, wherein the cannula delivery tool comprises a surface coating along the surface of the cannula delivery tool.

9. The percutaneous circulatory support system of claim 8, wherein the surface coating is silicone.

10. The percutaneous circulatory support system of claim 4, wherein a coefficient of friction between the cannula and the introducer sheath is greater than a coefficient of friction between the cannula delivery tool and the introducer sheath.

11. A cannula delivery tool configured for delivering a cannula, the cannula delivery tool comprising:
a proximal portion opposite a distal portion and a body portion extending therebetween, wherein the body portion comprises a tapered portion;
a curved plate extending from the proximal portion configured for easing the introduction of the cannula delivery tool into a sheath; and
a plurality of closed cells along the body portion formed by laser cutting a tube that forms the cannula delivery tool.

12. The cannula delivery tool of claim 11, wherein a tether is coupled to the curved plate and extends proximally from the curved plate of the cannula delivery tool, and wherein the tether is welded to the curved plate.

13. The percutaneous circulatory support system of claim 11, wherein the cannula delivery tool is composed of one of nitinol and stainless steel, and wherein the cannula delivery tool comprises a surface treatment.

14. The percutaneous circulatory support system of claim 11, wherein the cannula delivery tool is configured to compress from an expanded configuration having an expanded outer diameter to a compressed configuration have a compressed outer diameter.

15. A method of deploying a percutaneous support device, comprising:
providing a percutaneous support system including the percutaneous support device having at least an impeller housing coupled to a cannula, a handle for actuation of the percutaneous support system, a starter tube loaded on a catheter of the percutaneous support system, a cannula delivery tool loaded onto the catheter, and an introducer sheath;
extending the cannula delivery tool over the cannula;
retracting the cannula delivery tool and the percutaneous support device into the starter tube;
inserting the starter tube at least partially into the introducer sheath;
extending the cannula delivery tool out of a distal portion of the starter tube and at least partially out of a distal portion of the introducer sheath; and
extending the cannula out of the cannula delivery tool.

16. The method of claim 15, wherein retracting the delivery tool into the starter tube compresses the cannula delivery tool such that the cannula delivery tool comprises an outer diameter that is less than an inner diameter of the introducer sheath.

17. The method of claim 15, wherein the percutaneous support system further includes a guidewire, and wherein the method further includes extending the guidewire through the cannula delivery tube and the percutaneous support device prior to extending the cannula delivery tool over the cannula.

18. The method of claim 17, wherein the percutaneous support system further comprises a tether coupled to the introducer sheath and the cannula delivery tool, such that a length of the tether dictates how far the cannula delivery tool extends out of the introducer sheath.

19. The method of claim 15, wherein the method further comprises retracting the tether in order to retract the cannula delivery tool through the introducer sheath and into the starter tube.

20. The method of claim 16, wherein the cannula delivery tool comprises a tapered portion and a surface treatment, both configured to allow the cannula delivery tool to compress when retracted into the starter tube.
